(19) United States
(12) Patent Application Publication
(10) Pub. No.: US 2011/0130310 A1
(43) Pub. Date: Jun. 2, 2011

(54) MICROBIOREACTOR AND MICROTITER PLATE COMPRISING A PLURALITY OF MICROBIOREACTORS

(75) Inventors: Andreas Schober, Furth (DE); Caroline Augspurger, Ilmenau (DE); Frank Weise, Ilmenau (DE); Uta Fernekorn, Erfurt (DE); Christian Hildmann, Erfurt (DE); Jorg Hampl, Erfurt (DE)

(73) Assignee: TECHNISCHE UNIVERSITAT ILMENAU, Ilmenau (DE)

(21) Appl. No.: 12/675,225

(22) PCT Filed: Mar. 31, 2009
(86) PCT No.: PCT/EP09/53789

§ 371 Date: Aug. 5, 2010

(30) Foreign Application Priority Data
Apr. 3, 2008 (DE) 10 2008 017 765.2

(51) Int. Cl. C40B 60/12; C12M 3/00; C12M 1/34

(52) U.S. Cl. 506/39; 435/289.1; 435/287.1

(57) ABSTRACT

The present invention relates to a microbioreactor comprising a sample support (7) for holding cells; a bypass (3) with a capillary opening for feeding test reagent and/or for aeration and ventilation of the microbioreactor; a pump module (5) for perfusion of the microbioreactor with test reagent and/or gas feeding and purging of the microbioreactor; a transparent viewing window (2); and fluid channels (4) that form a circuit. The invention further relates to a bioreactor-microtiter plate comprising a plurality of such microbioreactors.

The invention relates to microfluidic systems which are preferably to be arranged in parallel and which are in general to be manufacture cost advantageously, for the cultivation of advanced cell cultures (3D culture, stem cells, etc.) which can be used advantageously in any laboratory working in biotechnology and biomedicine, and which is moreover HTS capable (HTS: High Throughput Screening). Such systems are in principle appropriate for use in the search for active ingredients in ADME/Tox screenings, and their marketing potential is consequently high. ADME/Tox studies are used to examine substances for their properties in the (human) organism with regard to Absorption, Distribution, Metabolization, Excretion and Toxicity. In particular, the invention relates to a microbioreactor with a sample support which carries, for example, a cell culture, and through which a fluid flows. A microbioreactor according to the invention is one which contains (in the static state) a medium quantity in the lower milliliter range, preferably in the microliter range. In addition, the invention relates to a CellChip arrangement or a microtiter plate, in which several microbioreactors are integrated.

In numerous applications in medicine and pharmaceutical research, or in the cosmetic industry, the cultivation of biological materials is a crucial task. In this context, to the extent possible, one should culture the cell material, which can be tissue sections, for example, from biopsies or from primary cells that were collected from animals or humans, cell lines, or genetically modified cells, in such a way that their natural functional and living capacities are maintained, or the desired functions are implemented as optimally as possible.

The bandwidth of application here ranges from the preparation of test systems for pharmaceutical and cosmetic research to medical research (for example, stem cell research), and to the production of vaccines. An important aspect here relates both to saving valuable cell material, and also to allowing many different parallel runs for testing different parameters.

In a tissue, the nutrients and substances for differentiation (messenger substances) must undergo intercellular introduction and removal not only by diffusion, but also with the help of active transport mechanisms, analogous to those in the blood circulation (and also the lymph system). Here it has been found that the geometry, for example, of support systems, frameworks for cell colonization, the separation and the guidance of fluidic supply channels, as well as the density, the species and the type of the cell colonization constitute important parameters that are decisive in tissue engineering with regard to the successful cultivation or manufacture of tissues. In addition, the induction of differentiation processes by the targeted release of active ingredients in a tissue remains an unsolved problem which to date has been approached only via “trial and error” methods.

For the proper functioning of a tissue or organ, many parameters are of significance. To ensure the maintenance of the organ and tissue functions in vitro, it is not only necessary to reproduce the correct molecular architecture, but also above all the correct macroscopic architecture of the cell aggregation. Thus, it is known that primary cells frequently lose their cell type specific (differentiated) functions when they are cultured in a monolayer (single layer). Therefore, various efforts have been made to develop cell culture systems that better reproduce the three-dimensional in vivo situation of the corresponding tissue.

One possibility is the microfluidically supported cell cultivation of cells in monolayer culture—preferably in 3D culture on different supports . Here, one starts with 3D cultures in microstructured supports (3D CellChips), which are already in the test stage, including for long-term cultivation. A 3D cell culture sample support here denotes a two-dimensionally or three-dimensionally constructed support structure for the three-dimensional cultivation of cells, which are preferably perforated, allowing medium to flow through them.

Such a sample support (3D CellChip) is known, for example, from WO 93/07258, where the orders of magnitudes for the support framework in advanced cell culture are based on physiological parameters. The height of the support framework for cells in a three-dimensional cultivation which is supplied from both sides should not exceed 300 μm, if there is no active flow through the cell layer. The prototype of the 3D CellChips accommodates, on a surface area of 1 cm², approximately 900 (30×30) microcontainers having the dimensions 300 μm×300 μm×300 μm (L×B×H) and a wall thickness of 50 μm. The bottom has pores that ensure unimpeded substance exchange already in case of superfusion, but also the flow of medium through the cell aggregation (perfusion).

The use of microsystem techniques for the manufacture of bioreactors has been found to be very advantageous for the defined cultivation of advanced cell cultures. The experimentally needed diversity with regard to different cell lines, serums, media, and active ingredients can be addressed economically only by the use of miniaturized systems.

From the dissertation by C. Augspurger (Leipzig 2007), it is known that such 3D CellChips (i.e., sample supports with 3D culture) are operated in accordance with standards, in bioreactors with several 25 mL volumes using external pumps as self-enclosed systems. However, these bioreactors can be used only limitedly for some applications, including in pharmaceutical research, because they are not compatible with the interfaces of automated laboratory technology, such as, for example, with the microtiter plate format (or also “MTP footprint”). In addition, it is not possible to introduce test substances automatically in these bioreactors. Moreover, they are characterized by an insufficient capacity to allow parallel runs. An additional problem consists of the fact that the required biological material or the volume used is simply too large for many tasks for these bioreactors to be used advantageously for parallel runs.

For the desired miniaturization and parallelization on an MTP footprint, the format, volume or the construction of the bioreactors known from the state of the art is too large and expensive, particularly for screening applications. From Wintermantel: Three-dimensional cell cultures; TUM Mitteilungen, 4-2006 (October 2006), it is known to introduce 3D structured polymers or polymer foams into 24- or 96-well MTP as support structures for 3D cultivation as passive systems. In contrast to the bioreactors that are known to date, these systems, however, have no active perfusion, and no active flow through the tissue and cell material, and thus represent an unsatisfactory model for imitating organs in the living organism.

In US 2007/0128715 A1, a perfusion device is described, which has a sample support for the reception of cells. In addition, inlet channels are present, through which the cells can be supplied with a substrate. An external pump can be connected to reinforce the perfusion. Because of the required external units, a parallel arrangement of numerous perfusion devices in an integrated unit, particularly in the format of a microtiter plate, is not possible.

US 2004/0229349 A1 describes, among other items, a device for the micro fluidic treatment and detection of particles, such as, for example, cells. The flow through several chambers is produced, for example, by a common peristaltic pump unit which is however not explained in greater detail. The cell chambers are in fluidic contact, so that a completely independent cultivation of different cell cultures in parallel cell chambers is not possible.

The problem of the present invention therefore is to overcome the disadvantages known from the state of the art, and to produce a microbioreactor as well as a miniaturizable, parallel bioreactor system for 3D cell cultivation for laboratory automation or for automated High Content Screening (HCS), i.e., for the automatic determination of numerous biological and physical parameters, or for the so-called High Throughput Screening (HTS).

According to the invention, this problem is solved with a microbioreactor according to claim 1 or a bioreactor microtiter plate according to claim 15.

A microbioreactor according to the invention consists substantially of a sample support for receiving cells; a bypass with a capillary opening for the addition of test reagent and/or for the ventilation or purging of the microbioreactor; an integrated pump module for the perfusion of the microbioreactor with test reagent and/or gas feeding or purging of the microbioreactor; a transparent viewing window and fluid channels that form a circuit.

The bioreactor microtiter plate according to the invention consists of a plurality of such microbioreactors.

Advantageous embodiments of the invention are indicated in the dependent claims.

The invention is explained in greater detail below in reference to the drawing. The figures in the drawing show:

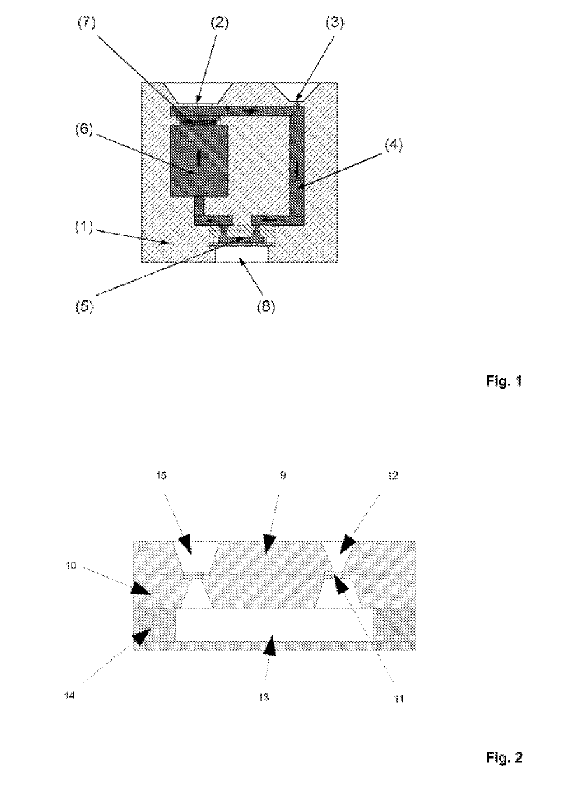

FIG. 1—a basic construction of a microbioreactor according to the invention;

FIG. 2—a basic construction of a membrane pump;

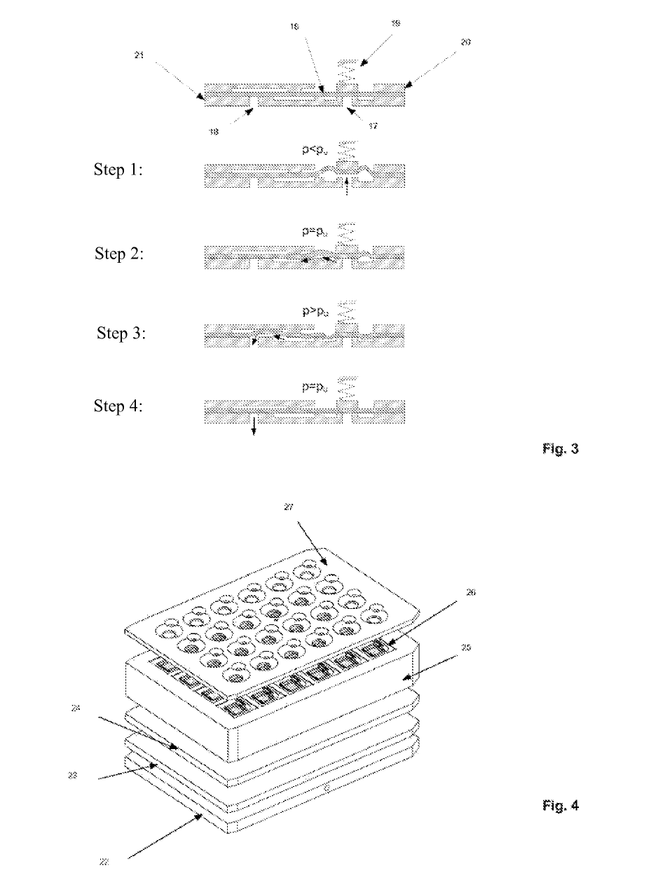

FIG. 3—several views of a peristaltic pump to illustrate the operation in several steps;

FIG. 4—an exploded perspective representation of the basic construction of a preferred bioreactor or CellChip microtiter plate according to the invention.

Below, two possible solution variants for the construction of microbioreactors with integrated microperforated support structures are differentiated: active systems, and passive systems which differ from the former. The term active system denotes systems in which a fluid is pumped through the system by means of a pump that is integrated in the system, and thus there is flow through a CellChip structure or the sample cultured therein. Passive systems, on the other hand, denote systems in which CellChip structures or samples are introduced into the microbioreactors or cultivation vessels, and flows—if there are any at all—are achieved by means of external pump systems, etc.

FIG. 1 represents a microbioreactor 1 according to the invention in a basic embodiment. The microbioreactor 1 consists of an integrated pump module 5, which can be designed, for example, as a membrane or peristaltic pump, a media reservoir 6, and a sample support 7 (preferably a 3D CellChip) with a sample which can be viewed from above through a transparent window 2. Because of the small distance between the window 2 and the sample, viewing under the microscope with small separation from the lens is possible. Via a bypass 3, a test reagent can be introduced from outside into the system through a fluid channel 4. The fluid channel 4 forms a circuit through the pump module 5 and the reservoir 6, through which circuit the fluid/test reagent is conveyed, to flow through the samples on the sample support 7. Via a feed line 8, a pump model 5 which is integrated in the microbioreactor can be operated, where a pneumatic activation or gas feeding or purging are possible. During the operation of the pump module 5, the pump conveys the fluid in the circuit, so that the cell culture (sample) can be supplied optimally with the nutrient medium or the test reagent.

In a variant embodiment, it is preferred, for use in cocultivation, to introduce two CellChips that are stacked on top of each other into the reactor.

The sample support 7 allows the two- or preferably three-dimensional cultivation of suspended or aggregated units, preferably cells or tissues. The sample support can for this purpose be designed as a retention membrane for suspended cells or as a cell culture substrate for adhering cells. For adhering cells, the sample support is preferably filled over the entire surface area, and not only partially, because in the latter case there would be more flow around instead of the desired flow through the filled surface area.

In an embodiment—represented in a simplified version in FIG. 2—of the microbioreactor according to the invention, a membrane pump is provided as pump module 5. It consists of a valve block made of two identical individual parts 9 and 10, which, depending on the material used, can also be designed to be inseparable, where, to ensure the functioning of the non-return valves, a special coating can be applied in each case on a valve seat 11, which prevents joining at this place. Through an inlet 12, the fluid is sucked in when a pump chamber 13 becomes larger as a result of a deformation of a work membrane of a chamber housing 14. During the subsequent reduction in the size of the chamber volume, the fluid is again conveyed out through the outlet 15. In near static operation, this pump has only one direction of conveyance, which is predetermined by the arrangement of the valves. The pump module 5 is integrated in such a way in the microbioreactor that a parallel or matrix-like arrangement of numerous microbioreactors in each case with integrated pump module, remains possible.

In case of an appropriate selection of the valve flaps, the chamber sizes, the membrane and the fluid, the above-mentioned pump can pump in the forward or backward direction as a function of the control frequency. An additional advantage of the use of a membrane consists in the active gas feeding into the medium, because the membrane material can be chosen in such a way that it is permeable to gases. The chamber volume of the membrane pump is a function of the desired rate of conveyance, and, in the microbioreactors according to the invention, it is preferably smaller than 2 μL.

In an additional embodiment of the microbioreactor according to the invention, a peristaltic pump is provided as pump module 5. The pump cycle of the peristaltic pump can be divided roughly into four sections which are represented in FIG. 3. First, a low pressure is applied via a work membrane 16. As a result, an inlet 17 of the pump opens, and the low pressure generated in the pump chamber is compensated by the inflow of the fluid to be conveyed. When the pressure applied to the work membrane 16 has reached the environmental pressure, the valve at the inlet 17 is closed again. As the pressure on the work membrane 16 continues to increase, the medium that has been sucked in previously flows in the direction of an outlet 18, because no other possibility exists for the medium due to the displacement. In the last section, the starting state exists again, i.e., at the outlet 18, the valve has closed again due to elastic effects. For this type of pump to work optimally, it is operated in the vicinity of the resonance frequency. Due to the phase change during the pass through the resonance frequency, the pump direction can also be reversed.

To overcome the limitation of the bioreactors and other systems known from the state of the art, in a parallel format according to the invention, each well, i.e., each microbioreactor, is provided with a micropump, which, in terms of its construction as a pulsating system, optionally reproduces the situation as found in an actual organism (beating heart, blood circulation).

Below, such a system is referred to as a “CellChip microtiter plate” (CellChip MTP) or “bioreactor microtiter plate” (BR MTP). A CellChip microtiter plate consists of an array of preferably mutually independent microbioreactors that are designed as active systems. The core of such a CellChip microtiter plate is at least one bioreactor with integrated pump preferably equipped with a CellChip as support for an advanced cell culture. Here the size of an individual reactor on a BR MTP is determined by the grid measure of a microtiter plate, in which 6, 12, 24, 48, 96 or more reactors are arranged in parallel, depending on the design. The volume of the media located inside an individual bioreactor varies thus as a function of the quantity of the reactors between 100 μL and 25 mL, but it is preferably in the lower milliliter range. All the channels preferably present a maximum cross section of less than 4 mm².

An unsolved problem encountered in all closed systems relates to the automatic introduction of test reagent into the system. It is precisely in parallelized arrangements of the MTP footprint, that the use of a pipetting robot is advantageous; however, in closed systems, this is possible only to an unsatisfactory degree. In a closed fluidic system, it is normally not possible to add fluid by dosing through contact place that is exposed. The fluid system is connected via a capillary, a kind of bypass, to the environment. This capillary allows the addition of small quantities of fluid. This is not expected to work in a completely flooded hydraulic system. Because this system has elastic elements, it can take up fluid to a certain degree. The quantity of the fed fluid depends on the size of the capillaries and on the maximum deformation of the elastic elements, i.e., the inner pressure of the system is limited by the boundary surface pressure of the medium with respect to the environment. Elastic elements in the sense of the invention can be produced by manufacturing the entire system from an elastic material (for example, silicon). The membrane of the membrane pump or the purposeful introduction of zones with very small wall thicknesses can also be considered to be an elastic element in this connection. Moreover, a purposeful surface structuring leads to elastic behavior of the system.

The capillary at its maximum dimension is within the order of magnitude of the fluid channels. In a partially sterile and nonsterile environment of the BR MTP, a septum or sterile filter can be used to close the capillary. Here the septum must allow, in terms of its size, the use of common commercial injection cannulas. In the case of separation by means of a sterile filter, the dimensions of the latter are also determined by the dimension of the fluid channels. The filter material must present a contact angle with respect to the test reagent of less than 90°, and a contact angle with respect to the medium of preferably more than 90°. A filter in the sense of the present invention must be a sieve structure that prevents the introduction of contaminating particles (for example, bacteria, cells, fungi, dust, . . . ). In a sterile environment of the BR MTP, the capillary opening can also be unclosed. Here, the dimension of the capillary opening should be chosen within the order of magnitude of the channels, but preferably in a range <500 μm. A capillary must be dimensioned in such a way that the inner pressure of the system (caused by a pump) is always smaller than the curvature pressure of the meniscus that develops on the capillary. The covering of the capillary with a simple cover is a part of the BR MTP according to the invention.

According to the invention, based on the construction, highly integrated, parallel systems with active elements for the perfusion of 3D cell cultivation systems can be produced in different embodiments preferably with integrated sensors (pH, O₂, CO₂, . . . ), and active systems on HTS installations, i.e., analogously to MTPs.

The first experimental setups focused on a 24-well reactor system. Such an active system is potentially capable of being transported, and it is designed in an encapsulated form, i.e., as a CellChip MTP with integrated fluidics with at first 24 independent bioreactors each with an opening and a bypass for the feeding of, for example, test reagents, and their distribution in the individual reactor.

In one embodiment, this should allow the transport of eukaryotic cells from the cell culture laboratory to the user. In this case, the cells are not deep frozen, but they remain in their culture environment for optimal cultivation in a module that is inserted into the transport box. The transport of simple cells is possible both in simple passive and also in active systems. For this purpose, in the simplest case, the 3D CellChips (samples) are housed, and gas fed with the appropriate carbogen mixture (O₂ with 5% CO₂), and preferably kept at a desired temperature to be set.

In an additional embodiment, the complete array can be manufactured and produced in a simplified manufacturing process. Such an embodiment is shown in FIG. 4. This embodiment of the bioreactor microtiter plate according to the invention is an active 24-well plate, consisting of a base plate 22 with connection for the pressure supply. A distribution plate 23 contains channels for the distribution of the driving pressurized air onto the 24 channels. A pump holder 24 receives the 24 micropumps for the individual wells. A grid plate 25 takes up several supports 26 for the CellChips. Each support 26 represents a circulation system of the microbioreactor, which functions to receive samples. Each circulation system comprises the sample support 7, the fluid channels 4 and optionally the media reservoir 6. The grid plate and the circulation systems can also be combined to a single component. The system is closed by means of a cover 27, in which the viewing window 2 and the accesses to the bypasses 3 are provided.

The complete CellChip MTP can accordingly also be produced from up to five unitary molded bodies, where intermediate steps can naturally be carried out, in which, for example, the membranes of the individual pump or the pumps are placed as individual elements in a frame.

In an additional advantageous embodiment, either by chemical or physical surface modification of the fluid channels, the wetting with the fluid is optimized in such a way that gas bubbles have no possibilities for adhesion there, or in such a way that no gas bubbles can adhere due to geometric minimization of relevant boundary surfaces. Conversely, it is possible to define places in the system that are precisely capable of functioning as bubble traps.

The integration of flow sensors can be implemented in an additional embodiment.

Cover slips to cover the bioreactors can be designed advantageously as optically transparent sensors, made preferably of the AlGaN/GaN material. The viewing window consists preferably of a transparent sensor material and functional material, particularly of materials of the group III-V semiconductors.

### List of Reference Numerals

1—Microbioreactor
2—Transparent window
3—Bypass with capillary opening
4—Fluid channel
5—Pump module
6—Media reservoir
7—Sample support
8—Feed line
9, 10—Identical components of a membrane pump
11—Valve seat in the membrane pump
12—Inlet of the membrane pump
13—Pump chamber of the membrane pump
14—Chamber housing with work membrane
15—Outlet of the membrane pump
16—Work membrane of a peristaltic pump
17—Inlet of the peristaltic pump
18—Outlet of the peristaltic pump
19—Resetting spring
20—Pump cover
21—Pump body
22—Base plate with connection for a pressure supply
23—Distribution plate
24—Pump holder
25—Grid plate
26—CellChip carrier/circulation system
27—Cover

1. A microbioreactor comprising:
(a) a sample support for receiving a sample comprising cells or tissues;
(b) an integrated pump module for perfusing the microbioreactor with a medium;
(c) fluid channels connected to the pump module and the sample support which fluid channels establish a circuit for the medium;
(d) a bypass with a capillary opening connected to the circuit of the fluid channels; and
(e) a transparent viewing window.

2. A microbioreactor according to claim 1, further comprising a media reservoir which is incorporated in the circuit.

3. A microbioreactor according to claim 1, wherein the transparent viewing window and the bypass are each on a top side of the microbioreactor.

4. A microbioreactor according to claim 1, wherein the sample support is (a) a single 3D CellChip or (b) several 3D CellChips stacked one on top of the other.

5. A microbioreactor according to claim 1, wherein the pump module is a peristaltic pump with an inlet or outlet and a work membrane for reversal of pump direction.

6. A microbioreactor according to claim 1, wherein the pump module is a membrane pump with two non-return valves and an inlet or outlet.

7. A microbioreactor according to claim 1, wherein the pump module comprises a permeable work membrane.

8. A microbioreactor according to claim 1, wherein the pump module comprises a feed line through which an operating means is supplied for the actuation of the pump module.

9. A microbioreactor according to claim 8, wherein the feed line opens at a bottom side of the microbioreactor.

10. A microbioreactor according to claim 1, wherein the surfaces of the fluid channels comprise a minimized boundary surface area.

11. A microbioreactor according to claim 1, further comprising an air bubble trap under and/or over the sample support, wherein the trap comprises a channel leading directly into it.

12. A microbioreactor according to claim 11, wherein the channel leading to the air bubble trap comprises a closure which is a septum.

13. A microbioreactor according to claim 1, wherein the transparent viewing window comprises one or more optically transparent sensors.

14. A microbioreactor according to claim 13, wherein the optically transparent sensors are AlGaN sensors.

15. A bioreactor microtiter plate comprising a plurality of microbioreactors according to claim 1 arranged in a matrix pattern, wherein the microbioreactors comprise circuits formed by fluid channels, which are independent of each other.

16. A bioreactor microtiter plate according to claim 15, wherein the plurality of microbioreactors are arranged in the grid of a microtiter plate and do not exceed its lateral external size.

17. A bioreactor microtiter plate according to claim 15, wherein the pump modules of all the microreactors each have their own feed line for supplying an operating medium.

18. A bioreactor microtiter plate according to claim 17, wherein the feed lines are through a common supply unit.

19. A bioreactor microtiter plate according to claim 15, comprising a system comprising a unit comprising formed parts comprising a base plate connected to a driven pressurized air feed, a distribution plate with channels for the distribution of the driving pressurized air, a pump holder for the reception of the individual pump modules, a grid plate for the reception of individual supports, and a cover, wherein the formed parts of each individual microreactor are distributed on the pump holder on the support in the grid plate, and on the cover.
